(19) United States
(12) Patent Application Publication
(10) Pub. No.: US 2012/0208245 A1
(43) Pub. Date: Aug. 16, 2012

(54) METHOD FOR PRODUCING L-LYSINE USING CORYNEBACTERIUM SP. THAT HAS OBTAINED THE ACTIVITY OF GLYCERALDEHYDE-3-PHOSPHATE DEHYDROGENASE DERIVED FROM AN ALIEN SPECIES

(75) Inventors: So Yeon Rah, Seoul (KR); Sang Jo Lim, Incheon (KR)

(73) Assignee: CJ CHEILJEDANG CORPORATION, Seoul (KR)

(21) Appl. No.: 13/382,899

(22) PCT Filed: May 28, 2010
(86) PCT No.: PCT/KR2010/003432

§ 371 Date: Apr. 24, 2012

(30) Foreign Application Priority Data
Jul. 8, 2009 (KR) 10-2009-0062322

(51) Int. Cl. C12P 13/08; C12N 1/21

(52) U.S. Cl. 435/115; 435/252.32

(57) ABSTRACT

The present invention relates to a Corynebacterium sp. strain having an activity of NADP-dependent glyceraldehyde-3-phosphate dehydrogenase and an improved productivity of L-lysine, and a method for producing L-lysine using the same. According to the Corynebacterium sp. strain of the present invention and the method for producing L-lysine using the same, L-lysine can be produced in a high yield.

### BACKGROUND OF THE INVENTION

1. Field of the Invention

The present invention relates to a Corynebacterium sp. strain having an activity of NADP-dependent glyceraldehyde-3-phosphate dehydrogenase and an improved productivity of L-lysine, and a method for producing L-lysine using the same.

2. Description of the Related Art

Traditionally, Coryneform bacteria are industrial microorganisms which are most widely used for the production of a variety of chemical materials useful in the animal feed, medicine and food industries, including amino acids, such as L-lysine, L-threonine, L-arginine, L-threonine and glutamic acid, and nucleic acid-related materials. These microorganisms are Gram-positive and require biotin for their growth. Representative examples of coryneform bacteria are the genus Corynebacterium including Corynebacterium glutamicum, the genus Brevibacterium including Brevibacterium flavum, the species Arthrobacter, the species Microbacterium, etc.

L-lysine is an L-amino acids, which is commercially used as a feed additive in animal nutrition due to its ability to help the body absorb other amino acids thereby improving the quality of feedstuff. For the human body, L-lysine is used as an ingredient of an injection solution, and also finds applications in the pharmaceutical field. Therefore, the industrial production of lysine is an economically important industrial process.

To improve the production yield of lysine, the enzyme activity on the lysine biosynthetic pathway has been typically enhanced by amplifying genes on the lysine biosynthetic pathway or by modifying promoter of the genes. In addition, an exogenous gene derived from other bacteria can be introduced, and for example, introduction of a gene coding for citrate synthase derived from Escherichia coli is described in Japanese Patent Publication No. Hei 7-121228.

Meanwhile, in vivo central carbon metabolic pathway can be modified in order to improve lysine productivity. Glyceraldehyde-3-phosphate dehydrogenase is an enzyme involved in the central carbon metabolic pathway. In Corynebacteria, it exists in a form of gapA which converts glyceraldehyde-3-phosphate into glycerate-1,3-bisphosphate using NAD as a coenzyme, in which glycerate-1,3-bisphosphate is converted into 3-phosphoglycerate by the pgk gene encoding enzyme. On the contrary, in streptococcus and Bacillus, it exists as a non-phosphorylating NADP-dependent GAPDH (non-phosphorylating NADP-dependent glyceraldehyde-3-phosphate dehydrogenase, hereinbelow, referred to as gapN) which converts glyceraldehyde-3-phosphate into 3-phosphoglycerate using NADP as a coenzyme, and produces NADPH. This process is a one-step process of playing an important role in glycolysis, and is affected by a NADPH/NADP ratio (Iddar, A et al., Protein Expr Purif. 25(3):519-26 (2002)).

It was reported that the enzyme gapN does not inherently exist in Corynebacterium. Also, there is no report of using the gapN gene in the culture of Corynebacterium. However, there are prior patents of increasing the amino acid productivity by introduction of the gapN gene. For example, Korean Patent Application No. 10-2004-0116999 and U.S. patent Ser. No. 11/722,820 describe that the gapN is expressed in E. coli to increase lysine productivity, but there is no report of applying it to Corynebacterium. Therefore, in order to achieve the effect of gapN gene, the Clostridium acetobutyricum-derived gapN was intended to be expressed in the lysine-producing Corynebacterium strain, but a satisfactory result could not be obtained. Thus, the present inventors have explored three types of gapN genes present in foreign bacteria through paper search (Abdelaziz Soukri et al. INTERNATIONAL MICROBIOLOGY (2005) 8:251-258)

The present inventors found that the gapN is expressed in order to use NADP, instead of NAD, as a coenzyme, and the resulting increase in NADPH levels is used as the source of reducing power for lysine biosynthesis, and thus the L-lysine productivity of Corynebacterium sp. strain can be enhanced, thereby completing the present invention.

### SUMMARY OF THE INVENTION

An object of the present invention is to provide a Corynebacterium sp. strain having an activity of NADP-dependent glyceraldehyde-3-phosphate dehydrogenase and an improved productivity of L-lysine, and a method for producing L-lysine using the same.

### BRIEF DESCRIPTION OF THE DRAWINGS

FIG. 1 shows a cleavage map of pECCG122-Pcj7-gapN, which is a expression plasmid for glyceraldehyde-3-phosphate dehydrogenase (gapN) of Streptococcus mutans, Streptococcus agalactiae, or Bacillus cereus; and

FIG. 2 shows a cleavage map of a PDZ-gapA chromosomal insertion vector for disruption of the gapA gene.

### DETAILED DESCRIPTION OF THE PREFERRED EMBODIMENTS

In one embodiment, the present invention provides a Corynebacterium sp. strain having an activity of NADP-dependent glyceraldehyde-3-phosphate dehydrogenase and an improved productivity of L-lysine.

The Corynebacterium sp. strain having an improved productivity of L-lysine of the present invention may be obtained by inactivating the endogenous glyceraldehyde-3-phosphate dehydrogenase-encoding gene (gapA) and introducing the exogenous NADP-dependent glyceraldehyde-3-phosphate dehydrogenase-encoding gene (gapN).

In the present invention, the exogenous NADP-dependent glyceraldehyde-3-phosphate dehydrogenase-encoding gene includes any nucleotide encoding a polypeptide, as long as the polypeptide has an activity of converting into 3-phosphoglycerate by using glyceraldehyde-3-phosphate as a substrate and NADP as a coenzyme.

Examples of exogenous NADP-dependent glyceraldehyde-3-phosphate dehydrogenase-encoding gene include NADP-dependent glyceraldehyde-3-phosphate dehydrogenase-encoding genes that are derived from animals, plants and bacteria, preferably, NADP-dependent glyceraldehyde-3-phosphate dehydrogenase-encoding genes that are derived from bacteria, and most preferably, an NADP-dependent glyceraldehyde-3-phosphate dehydrogenase-encoding gene that is originated from Streptococcus mutans, Streptococcus agalactiae, or Bacillus cereus. More specifically, the genes of Streptococcus mutans, Streptococcus agalactiae, and Bacillus cereus may have the nucleotide sequences of SEQ ID NOS. 11 (GenBank accession No. NC_{—}004350), 12 (GenBank accession No. NC_{—}004116), and 13 (GenBank accession No. NC_{—}004722.1), respectively.

The exogenous NADP-dependent glyceraldehyde-3-phosphate dehydrogenase-encoding gene may be introduced into a coryneform bacterium using a common method widely known in the art.

Specifically, it can be achieved by introducing the exogenous NADP-dependent glyceraldehyde-3-phosphate dehydrogenase-encoding gene into a vector so as to obtain a NADP-dependent glyceraldehyde-3-phosphate dehydrogenase-expressing recombinant vector; and introducing the recombinant vector into a coryneform bacterium having an inactivated endogenous glyceraldehyde-3-phosphate dehydrogenase gene so as to produce a transformed coryneform bacterium.

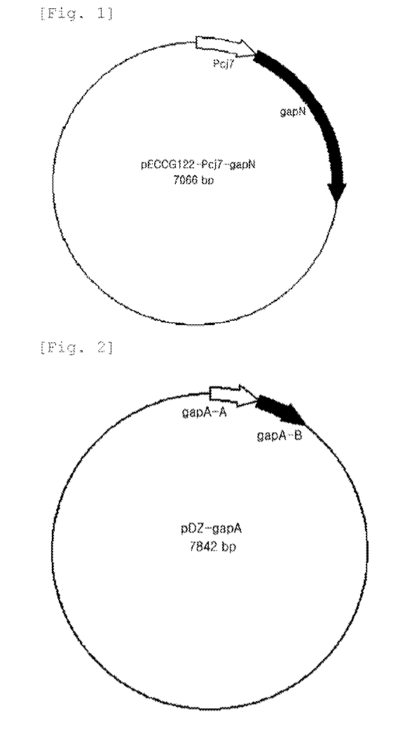

The NADP-dependent glyceraldehyde-3-phosphate dehydrogenase-expressing recombinant vector may be prepared by a common method, for example, ligating the sequence of the exogenous NADP-dependent glyceraldehyde-3-phosphate dehydrogenase gene to a suitable vector using a restriction enzyme. In the present invention, the vector may be a vector having the cleavage map of FIG. 1. In the specific embodiment of the present invention, the vectors for the introduction of the genes of Streptococcus mutans, Streptococcus agalactiae, and Bacillus cereus are pECCG122-Pcj7-gapN1, pECCG122-Pcj7-gapN2, and pECCG122-Pcj7-gapN3, respectively.

The endogenous glyceraldehyde-3-phosphate dehydrogenase-encoding gene refers to a gene encoding the enzyme that has an activity of converting into glycerate-1,3-bisphosphate by using glyceraldehyde-3-phosphate as a substrate and NAD as a coenzyme in the Corynebacterium sp. strain, and preferably refers to a gene having the nucleotide sequence of SEQ ID NO. 14 (NCBI Accession No. NC_{—}003450, NCg11526)

Further, in order to inactivate the endogenous glyceraldehyde-3-phosphate dehydrogenase-encoding gene, genetic manipulation of deletion, substitution or insertion of the native gapA gene of the Corynebacterium sp. strain may be performed. Preferably, a coryneform bacterium may be transformed with a recombinant vector including a part of the gapA gene, and for example, a strain having the inactivated gapA activity may be prepared by introduction of a vector having the cleavage map of FIG. 2. In the specific embodiment of the present invention, a pDZ-gapA recombinant vector having two fragments of a part of the endogenous glyceraldehyde-3-phosphate dehydrogenase-encoding gene consecutively cloned was used as a vector for chromosomal insertion. Preferably, a coryneform bacterium to be transformed with this vector for chromosomal insertion is, but not limited to, Corynebacterium glutamicum KFCC-10881. According to the specific embodiment of the present invention, the coryneform bacterium having the disruption of the endogenous glyceraldehyde-3-phosphate dehydrogenase-encoding gene may be Corynebacterium glutamicum CA01-0096 (Accession No. KCCM 11012P).

As used herein, the term “transformation” means that DNA is introduced into a host wherein the DNA remains as an extrachromosomal element or integrated into the chromosome and is made to be replicable. As used herein, the term “transfection” means that an expression vector having an arbitrary coding sequence which may be expressed or not is received by a host cell. As used herein, the terms “transfected host” and “transformed host” mean a cell into which DNA is introduced. The cell is called a “host cell”, and it may be a prokaryotic or eukaryotic cell. The typical prokaryotic cell includes a variety of strains such as E. coli. The term “vector” refers to a DNA product having a DNA sequence operably linked to a regulatory sequence that can express DNA in a suitable host. Examples of the regulatory sequence include a promoter for transcription, an arbitrary operator sequence for regulating transcription, a sequence encoding an appropriate mRNA ribosome binding site, and sequences for regulating the termination of transcription and translation. Examples of the vector may include plasmids, phage particles, or simply potential genomic inserts. Once transformed into a suitable host, the vector may replicate and function independently of the host genome, or may, in some instances, integrate into the genome itself. As used herein, “plasmid” and “vector” are sometimes used interchangeably as the plasmid is the most commonly used form of vector at present. The term “regulatory sequence” refers to a DNA sequence necessary for the expression of the operably linked coding sequence in a particular host organism. For example, regulatory sequences necessary for expression in prokaryotes include a promoter, optionally an operator sequence, and a ribosome binding site. Eukaryotes are known to use a promoter, a polyadenylation signal, and an enhancer.

When a nucleic acid is disposed in a functional relationship with other nucleic acid sequence, it is “operably linked” to the nucleic acid sequence. It means that genes are linked in such a manner as to enable expression of the gene when the regulatory sequence(s) couples with an appropriate molecule (e.g., a transcription-activating protein). For example, DNA of a pre-sequence or a secretory leader is operably linked to DNA of polypeptide when it is expressed as a pre-protein that participates in secretion of polypeptide; a promoter or enhancer is operably linked to a coding sequence when it affects transcription of the coding sequence; a ribosome binding site is operably linked to a coding sequence when it affects transcription of the coding sequence; or the ribosome binding site is operably linked to the coding sequence when it is disposed to facilitate translation. In general, “operably linked” means that DNA sequences being linked are contiguous, and in the case of the secretory leader, contiguous and in a reading frame. However, an enhancer does not have to contact a coding sequence. Linkage between sequences may be performed by ligation in a convenient restriction enzyme site. However, when there is no restriction enzyme site, a synthetic oligonucleotide adaptor or linker may be used according to an ordinary method.

As used herein, the term “vector” refers to a recombinant carrier into which heterologous DNA fragments are inserted, in which the DNA fragment is generally a double-strand DNA fragment. Here, heterologous DNA means hetero-type DNA that is not naturally found in a host cell. Once the expression vector is incorporated with a host cell, it can be replicated irrespective of host genomic DNA to generate several copies and their inserted (heterologous) DNAs.

As is known to one skilled in the art, in order to raise an expression level of a transfected gene in a host cell, the corresponding gene must be operably linked to an expression control sequence that performs transcription and translation functions in a selected expression host. Preferably, the expression control sequence and the gene are included in a single expression vector comprising both a bacterial selectable marker and a replication origin. When an expression host is a eukaryotic cell, the expression vector must further include an expression marker useful in the eukaryotic expression host.

The vector useful for the preparation of the recombinant vector of the present invention may be derived from vectors autonomically replicable in the coryneform bacteria, and for example, a phage vector or a plasmid vector. Examples of the phage vector or the cosmid vector include pWE15, M13, λEMBL3, λEMBL4, λFIXII, λDASHII, λZAPII, λgt10, λgt11, Charon4A, and Charon21A, and examples of the plasmid vector include a pBR plasmid, a pUC plasmid, a pBluescriptII plasmid, a pGEM plasmid, a pTZ plasmid, a pET plasmid, a pMal plasmid, a pQE plasmid, etc.

Further, the Corynebacterium sp. used in the present invention includes any Corynebacterium sp., as long as it has L-lysine productivity. Examples of the Corynebacterium sp. include Corynebacterium glutamicum, Corynebacterium thermoaminogenes, Brevibacterium flavum, and Brevibacterium fermentum.

The Corynebacterium sp. of the present invention includes a mutant strain having enhanced L-lysine productivity as well as a wild-type strain. Examples thereof include strains that nutritionally require methionine or are resistant to threonine analogues (AHV: α-amino-β-hydroxy valeric acid), lysine analogues (AEC: S-(2-aminoethyl)-L-cysteine), isoleucine analogues (α-aminobutyric acid), methionine analogues (ethionine), etc. In addition, in order to improve the L-lysine productivity, the strain may be a mutant having an improved L-lysine productivity that is prepared by increasing the copy number of the introduced gene or manipulating a gene regulatory sequence of the gene. Preferably, it may include Corynebacterium glutamicum ATCC13032, Corynebacterium thermoaminogenes FERM BP-1539, Brevibacterium flavum ATCC 14067, Brevibacterium lactofermentum ATCC 13869, and an L-amino acid-producing mutant or strain prepared therefrom, for example, Corynebacterium glutamicum KFCC11001, and most preferably, Corynebacterium glutamicum KFCC10881, but is not limited thereto.

The specific embodiment of the present invention provides Corynebacterium sp. strains of Corynebacterium glutamicum CA01-0565 (Accession No. KCCM 11013P), Corynebacterium glutamicum CA01-0566 (Accession No. KCCM 11014P), and Corynebacterium glutamicum CA01-0567 (Accession No. KCCM 11015P), which are prepared by introducing the gene of Streptococcus mutans, the gene of Streptococcus agalactiae, and the gene of Bacillus cereus into the Corynebacterium glutamicum KFCC10881 having the inactivated endogenous glyceraldehyde-3-phosphate dehydrogenase-encoding gene, respectively. These strains were deposited in KCCM (Korean Culture Center of Microorganisms, located at 361-221 Yurim Bild., Hongje 1-dong, Seodaemun-gu, Seoul, Korea) on Jul. 2, 2009.

The improvement in L-lysine productivity is attributed to the increased reducing power by activation of NADP-dependent glyceraldehyde-3-phosphate dehydrogenase. Instead of the endogenous glyceraldehyde-3-phosphate dehydrogenase, an exogenous NADP-dependent glyceraldehyde-3-phosphate dehydrogenase is introduced into the L-lysine-producing Corynebacterium strain, so that NADP-dependent glyceraldehyde-3-phosphate dehydrogenase is activated to make the strain to utilize NADP instead of NAD as a coenzyme, and thus the resulting increase in NADPH levels can be used as the source of reducing power for L-lysine biosynthesis.

Another embodiment of the present invention provides a method for producing L-lysine, comprising the steps of culturing the Corynebacterium sp. strain having an activity of NADP-dependent glyceraldehyde-3-phosphate dehydrogenase and an improved productivity of L-lysine, and recovering lysine from the cultured cells or the culture broth.

The Corynebacterium sp. strain used according to the present invention may be cultured by continuous or batch type method such as batch, fed-batch and repeated fed-batch cultures. A summary of known cultivation methods is described in the textbook [Chmiel, (Bioprozesstechnik 1. Einfuhrung in die Bioverfahrenstechnik (Gustav Fischer Verlag, Stuttgart, 1991); and Storhas (Bioreaktoren and periphere Einrichtungen (Vieweg Verlag, Braunschweig/Wiesbaden, 1994)].

The culture medium to be used must meet the requirements of the particular strains in a suitable manner. Descriptions of culture media for Corynebacterium sp. strain are to be found in the handbook [“Manual of Methods for General Bacteriology” by the American Society for Bacteriology (Washington D.C., USA, 1981)]. The useful carbon source may include sugars and carbohydrates such as glucose, saccharose, lactose, fructose, maltose, starch and cellulose, oils and fats such as soybean oil, sunflower oil, groundnut oil and coconut oil, fatty acids such as palmitic acid, stearic acid and linoleic acid, alcohols such as glycerol and ethanol, and organic acids such as acetic acid. Those substances may be used individually or in the form of a mixture. The useful nitrogen source may include organic nitrogen-containing compounds such as peptones, yeast extract, meat extract, malt extract, corn steep liquor, soybean flour and urea, or inorganic compounds such as ammonium sulfate, ammonium chloride, ammonium phosphate, ammonium carbonate and ammonium nitrate. The nitrogen sources may be used individually or in the form of a mixture. The useful phosphorus source may include potassium dihydrogen phosphate or dipotassium hydrogen phosphate or the corresponding sodium-containing salts. The culture medium must also contain metal salts such as magnesium sulfate or iron sulfate, which are necessary for growth. Finally, essential growth substances, such as amino acids and vitamins, may be used in addition to the above-mentioned substances. Suitable precursors may be also added to the culture medium. The mentioned substances may be added to the culture by continuous or batch type in a suitable manner during the cultivation.

In order to control the pH value of the culture, basic compounds such as sodium hydroxide, potassium hydroxide, and ammonia or acid compounds such as phosphoric acid or sulfuric acid, are expediently used. In order to control the development of foam, an anti-foaming agent such as fatty acid polyglycol esters may be used. In order to maintain aerobic conditions, oxygen or oxygen-containing gas such as air is introduced into the culture. The temperature of the culture is normally from 20° C. to 45° C., and preferably from 25° C. to 40° C. The culture is continued until the maximum amount of the desired L-amino acids has formed. That aim is normally achieved within a period of from 10 hours to 160 hours.

The analysis of L-amino acids may be carried out by anion-exchange chromatography with subsequent ninhydrin derivatization (Spackman et al. (Analytical Chemistry, 30, (1958), 1190).

The present invention will be described in more detail with reference to the following Examples. However, the invention is not intended to be limited by these Examples.

### Example 1

### Exploration and Cloning of gapN Gene of Streptococcus mutans

The sequence of the Streptococcus-derived gapN gene has been clearly revealed. The gapN gene information of Streptococcus mutans (accession No. NC_{—}004350) was acquired from NIH GenBank. Based on the reported sequence, a pair of primers described in the following Table 1 was synthesized and 1428 base pairs of the gapN gene were amplified by Polymerization Chain Reaction (PCR) [Sambrook et al, Molecular Cloning, a Laboratory Manual (1989), Cold Spring Harbor Laboratories](PCR conditions: Denaturing=94° C., 30 sec/Annealing=50° C., 30 sec/Polymerization=72° C., 1 min 30 sec, 30 cycles) using a chromosomal DNA of the Streptococcus mutans ATCC25175 strain as a template, and cloned into an E. coli plasmid pCR2.1 using a TOPO Cloning Kit (Invitrogen) so as to obtain a pCR-gapN1 plasmid.

TABLE 1
Primer

| | | SEQ ID |
|---|---|---|
| Primer | sequence | NO. |
| gapN_F1 | 5′-GCG CAT ATG ACA AAA CAA TAT | 1 |
| | AA AAA-3′ | |
| gapN_R1 | 5′-GCG TCT AGA TTA TTT GAT ATC | 2 |
| | AAA TAC-3′ | |

### Example 2

### Construction of gapN Expression Vector

The gapN gene-containing pCR-gapN1 vector obtained in Example 1 was cleaved using restriction enzymes Nde-I and Xba-I, and the gapN-encoding gene fragment was only separated, and an expression promoter Pcj7 (reference; Patent Publication No. 2006-0068505) was ligated with a shuttle vector pECCG122 of E. coli and Corynebacterium (reference; Patent Publication No. 1992-0000933), so as to construct pECCG122-Pcj7-gapN1 (FIG. 1).

### Example 3

### Exploration and Cloning of gapN Gene of Streptococcus agalactie

The sequence of the Streptococcus-derived gapN gene has been clearly revealed. The gapN gene information of Streptococcus agalactie (accession No. NC_{—}004116) was acquired from NIH GenBank. Based on the reported sequence, a pair of primers described in the following Table 2 was synthesized and 1428 base pairs of the gapN gene were amplified by Polymerization Chain Reaction (PCR) [Sambrook et al, Molecular Cloning, a Laboratory Manual (1989), Cold Spring Harbor Laboratories](PCR conditions: Denaturing=94° C., 30 sec/Annealing=50° C., 30 sec/Polymerization=72° C., 1 min 30 sec, 30 cycles) using a chromosomal DNA of the Streptococcus agalactie ATCC BAA-611 strain as a template, and cloned into an E. coli plasmid pCR2.1 using a TOPO Cloning Kit (Invitrogen) so as to obtain a pCR-gapN2 plasmid.

TABLE 2
Primer

| | | SEQ |
|---|---|---|
| | | ID |
| Primer | sequence | NO. |
| gapN_F2 | 5′-GCG CAT ATG ACA AAA GAA TAT | 3 |
| | CAA-3′ | |
| gapN_R2 | 5′-GCG TCT AGA CTA TTT CAT ATC AAA | 4 |
| | AAC-3′ | |

### Example 4

### Construction of gapN Expression Vector

The gapN gene-containing pCR-gapN 2 vector obtained in Example 3 was cleaved using restriction enzymes Nde-I and Xba-I, and the gapN-encoding gene fragment was only separated, and an expression promoter Pcj7 (reference; Patent Publication No. 2006-0068505) was ligated with a shuttle vector pECCG122 of E. coli and Corynebacterium (reference; Patent Publication No. 1992-0000933), so as to construct pECCG122-Pcj7-gapN2 (FIG. 1).

### Example 5

### Exploration and Cloning of gapN Gene of Bacillus cereus

The sequence of the Bacillus-derived gapN gene has been clearly revealed. The gapN gene information of Bacillus cereus (accession No. NC_{—}004722.1) was acquired from NIH GenBank. Based on the reported sequence, a pair of primers described in the following Table 3 was synthesized and 1428 base pairs of the gapN gene were amplified by Polymerization Chain Reaction (PCR) [Sambrook et al, Molecular Cloning, a Laboratory Manual (1989), Cold Spring Harbor Laboratories](PCR conditions: Denaturing=94° C., 30 sec/Annealing=50° C., 30 sec/Polymerization=72° C., 1 min 30 sec, 30 cycles) using a chromosomal DNA of the Bacillus cereus ATCC 14579 strain as a template, and cloned into an E. coli plasmid pCR2.1 using a TOPO Cloning Kit (Invitrogen) so as to obtain a pCR-gapN3 plasmid.

TABLE 3
Primer

| | | SEQ ID |
|---|---|---|
| Primer | sequence | NO. |
| gapN_F3 | 5′-GCG CAT ATG ACA ACT AGC AAT | 5 |
| | ACG-3′ | |
| gapN_R3 | 5′-GCG TCT AGA TTA AAC TAA GTT | 6 |
| | TAA-3′ | |

### Example 6

### Construction of gapN Expression Vector

The gapN gene-containing pCR-gapN3 vector obtained in Example 5 was cleaved using restriction enzymes Nde-I and Xba-I, and the gapN-encoding gene fragment was only separated, and an expression promoter Pcj7 was ligated with a shuttle vector pECCG122 of E. coli and Corynebacterium, so as to construct pECCG122-Pcj7-gapN3 (FIG. 1).

### Example 7

### Preparation of Lysine-Producing Corynebacterium glutamicum (KFCC-10881) by Artificial Mutation

Corynebacterium glutamicum (KFCC-10881) prepared by artificial mutation, which is resistant to S-(2-aminoethyl) cysteine (hereinafter referred to as “AEC”) and is homoserine-leaky, was used as a strain into which a plurality of genes responsible for lysine biosynthetic pathway were to be inserted.

The mutant strain KFCC-10881 was prepared from the wild-type Corynebacterium glutamicum (ATCC13032) as a mother strain. The mother strain of 10⁷⁻10⁸ cells/ml was treated with the mutagen N-methyl-N′-nitro-N-nitrosoguanidine (hereinafter referred to as “NTG”) in a final concentration of 500 μg/ml at 30° C. for 30 min, followed by the selection of colonies grown on a complex plate containing 5 g/l of AEC. After the primary mutant strain was analyzed for AEC resistance and lysine productivity, it was led to secondary mutation with NTG. A plurality of the colonies thus formed were tooth-picked into minimal media, which were or were not supplemented with homoserine (100 mg/L), so as to separate homoserine auxotrophs (secondary mutants), which cannot grow in a minimal medium lacking homoserine. The homoserine auxotroph was allowed to undergo tertiary mutation so as to create a homoserine-leaky strain which was identified by incubation in a minimal medium containing 10 mg/L of homoserine. The strain grown in the medium was examined for lysine productivity (Table 4). The resulting lysine-producing strain, which is AEC resistant and homoserine-leaky, was deposited with the Korean Federation of Culture Collection under the accession number of KFCC-10881.

TABLE 4
Lysine productivity of KFCC-10881

| | Lysine (g/l) | | |
|---|---|---|---|
| Strain | Batch 1 | Batch 2 | Batch 3 |
| Wild-type | 0 | 0 | 0 |
| (ATCC13032) | | | |
| KFCC-10881 | 45 | 43 | 42.5 |

Minimal Medium (pH 7.0)

Glucose 10 g, (NH₄)₂SO₄ 5 g, urea 2 g, KH₂PO₄ 1 g, K₂HPO₄ 2 g, MgSO₄ 7H₂O 0.4 g, biotin 200 μg, thiamine HCl 3000 μg, Calcium Pantothenate 1000 μg, nicotinamide 5000 μg, NaCl 0.5 g (per 1 liter of distilled water)

Seed Medium (pH 7.0)

Glucose 20 g, Peptone 10 g, Yeast Extract 10 g, urea 5 g, KH₂PO₄ 4 g, K₂HPO₄ 8 g, MgSO₄ 7 H₂O 0.5 g, biotin 100 μg, thiamine HCl 1000 μg (per 1 liter of process water)

Production Medium (pH 7.0)

Glucose 100 g, (NH₄)₂SO₄ 40 g, Soy Protein 2.5 g, Corn Steep Solids 5 g, urea 3 g, KH₂PO₄ 1 g, MgSO₄ 7H₂O 0.5 g, biotin 100 μg, thiamine HCl 1000 μg, CaCO₃ 30 g (per 1 liter of distilled water)

### Example 8

### Cloning of Lysine-Producing Corynebacterium glutamicum KFCC-10881-Derived gapA Gene, Construction of Recombinant Vector (pDZ-gapA), and Preparation of gapA-Disrupted Strain

In this Example, the gapA gene of lysine biosynthetic pathway was acquired by PCR using a chromosomal DNA of the lysine-producing Corynebacterium glutamicum KFCC10881 prepared in Example 7 as a template. Based on the NIH GenBank, sequence information of the gapN gene (NCBI accession No. NC_{—}003450, NCg11526) was acquired, and two pairs of primers (Table 5, SEQ ID NOS. 7 to 10) were synthesized.

PCR was performed using the chromosomal DNA of Corynebacterium glutamicum KFCC10881 as a template and a set of oligonucleotide primers of SEQ ID NOS. 7 and 10 in the presence of PfuUltra™ High-Fidelity DNA Polymerase (Stratagene), with 30 cycles of denaturing at 96° C. for 30 sec; annealing at 53° C. for 30 sec; and polymerization at 72° C. for 30 sec. The PCR products thus obtained were found to be two kinds of gapA gene fragment (gapA-A, gapA-B) of 600 bp. The gapA-A was amplified using the primers of SEQ ID NOS. 7 and 8, and the gapA-B was amplified using the primers of SEQ ID NOS. 9 and 10. The amplified products were cloned into an E. coli vector pCR2.1 using a TOPO Cloning Kit (Invitrogen), so as to obtain pCR-gapA-A and pCR-gapA-B vectors.

TABLE 5
Primer

| Primer | sequence | SEQ ID NO. |
|---|---|---|
| F- gapA -SalI_P1 | CAC GTC GAC GAA TGT GTC TGT ATG | 7 |
| R- gapA- XbaI_P2 | TGA TCT AGA AGA TGA TGA CCT TCT | 8 |
| F- gapA -XbaI_P3 | CCA TCT AGA GCT CTG GTT CTC CCA GAG | 9 |
| R- gapA -XbaI_P4 | GCT TCT AGA GGT CTT AAC AGC CAT GCC | 10 |

These pCR vectors were treated respectively with the restriction enzymes included in each end of gapA-A and gapA-B (gapA-A: SalI, XbaI, gapA-B: XbaI) to separate the gapA genes from the pCR vectors. Next, these fragments were cloned through 3-piece ligation into a pDZ vector (reference: Patent Publication No. 10-2008-0025355) treated with restriction enzymes SalI and XbaI, so as to produce a pDZ-gapA recombinant vector, in which two copies of gapA were consecutively cloned. FIG. 2 shows a pDZ-gapA vector for chromosomal insertion of Corynebacterium.

The constructed pDZ-gapA vector was transformed into the lysine-producing Corynebacterium glutamicum KFCC-10881 prepared in Example 7, followed by insertion of a vector having 300 base pairs deletion within the gapA gene into the gapA gene on the chromosome through secondary crossover, so as to produce a lysine-producing Corynebacterium glutamicum CA01-0096 having the inactivated gapA gene (Accession No. KCCM 11012P). PCR was performed using the primers of SEQ ID NOS. 7 and 10 to confirm that the disrupted gapA gene has 300 base pairs deletion of the wild-type gapA gene.

### Example 9

### Induction of gapN Gene Expression in CA01-0096

The pECCG122-Pcj7-gapN1˜3 vectors obtained in Examples 2, 4, and 6 were introduced into CA01-0096. The gapA-disrupted strain is not known to grow in a minimal medium where Corynebacterium glutamicum usually grows (reference paper; Hideaki Yukawa et al., J Mol Microbiol Biotechnol 2004; 8:91-103).

The pECCG122-Pcj7-gapN1˜3 vectors were transformed into CA01-0096 by an electrical pulse method so as to prepare transformed strains (Strain No. CA01-0565, CA01-0566, CA01-0567). The gapN expression was induced in Corynebacterium glutamicum by utilizing the characteristic of growing in a minimal medium by gapN expression. The CA01-0565, CA 01-0566, CA 02-0567 strains were deposited in KCCM (Korean Culture Center of Microorganisms, located at 361-221 Yurim Bild., Hongje 1-dong, Seodaemun-gu, Seoul, Korea) on Jul. 2, 2009 with Accession NOS. KCCM 11013P, KCCM 11014P, and KCCM 11015P, respectively.

### Example 10

### Examination of gapN Activity in Lysine-Producing Strain

Growth in a minimal medium using glucose as a carbon source was examined. As a result, the gapA-disrupted strain did not grow and the gapN-expressing strain recovered to grow.

TABLE 6
Growth

| Strain | Growth | No growth |
|---|---|---|
| KFCC10881 | Growth | |
| CA01-0096 | | No growth |
| CA01-0565 | Growth | |
| CA01-0566 | Growth | |
| CA01-0567 | Growth | |

### Example 11

### Examination of gapN Activity in Lysine-Producing Strain

The gapN expression levels were measured to confirm whether the gapN gene from the gapN expression vector was expressed in a cell so as to exhibit its activity. The CA01-0565, CA01-0566, and CA01-0567 strains were prepared by introducing each of the pECCG122-Pcj7-gapN1, 2, 3 vectors into the gapA-disrupted strain, the gapA-disrupted CA01-0096 strain, and the gapA-existing KFCC-10881 strain were cultured in seed media for one day, and then diluted at O.D 600=0.2, and 25 ml of each cell was recovered at O.D 600=10. The gapN activity was determined by a method described in A. Soukri et al., Protein Expression and Purification; 25; (2002) 519-529. As a result, it was found that gapN1 has an activity of 1 unit, gapN2 has an activity of 0.64 unit, and gapN3 has an activity of 0.09 unit (Table 7).

TABLE 7
Assay Result

| | Strain | | | | |
|---|---|---|---|---|---|
| | | | | | Bacillus |
| | | | Streptococcus | Streptococcus | cereus |
| | | ΔgapA | mutans | agalactie | gapN |
| | control | CA01- | gapN | gapN | CA01- |
| | KFCC10881 | 0096 | CA01-0565 | CA01-0566 | 0567 |
| gapN | 0 | 0 | 1 | 0.64 | 0.09 |
| activity | | | | | |
| (unit) | | | | | |

Unit; nmol/mg · min = unit

### Example 12

### Lysine Productivity of gapN-Expressing Strain

The L-lysine productivity of the CA01-0565, CA01-0566, and CA01-0567 strains was analyzed by the following method. Corynebacterium glutamicum KFCC-10881, CA01-0096, CA01-0565, CA01-0566, and CA01-0567 were inoculated into 250 ml-corner baffle flasks containing 25 ml of the seed medium and cultured at 30° C. for 20 hour with shaking (220 rpm). Thereafter, 1 ml of each of the seed cultures was inoculated into a 250 ml-corner baffle flask containing 25 ml of the production medium and cultured at 35° C. for 96 hours with shaking (200 rpm). After the completion of culture, HPLC analysis was performed to determine the amounts of the L-lysine produced by the strains. The concentrations of L-lysine in the cultures of Corynebacterium glutamicum KFCC-10881, CA01-0096, CA01-0565, CA01-0566, and CA01-0567 are summarized in the following Table 8. As a result, the gapA-disrupted strain did not produce lysine. It was found that lysine was produced by the gapN gene introduced into the gapA-disrupted strain, and the production amounts were also increased by 16˜17%, compared to that of the mother strain KFCC-10881 (Table 8).

TABLE 8
Flask culture result

| Strain name | Lysine (g/l) |
|---|---|
| KFCC-10881 | 42 |
| CA01-0096 | 0 |
| CA01-0565 | 50 |
| CA01-0566 | 48.8 |
| CA01-0567 | 46 |

### EFFECT OF THE INVENTION

According to the present invention, Corynebacterium strain having an L-lysine productivity and a gapN activity is prepared by introduction of Streptococcus and Bacillus-derived gapN-encoding genes, and thus the reducing power is increased to supply energy required for lysine production, leading to enhancement in the L-lysine production.

1. A Corynebacterium sp. strain having an activity of NADP-dependent glyceraldehyde-3-phosphate dehydrogenase and an improved productivity of L-lysine.

2. The Corynebacterium sp. strain according to claim 1, wherein the endogenous glyceraldehyde-3-phosphate dehydrogenase-encoding gene (gapA) is inactivated, and the exogenous NADP-dependent glyceraldehyde-3-phosphate dehydrogenase-encoding gene (gapN) is introduced.

3. The Corynebacterium sp. strain according to claim 2, wherein the exogenous NADP-dependent glyceraldehyde-3-phosphate dehydrogenase-encoding gene is originated from Streptococcus mutans (ATCC 25175), Streptococcus agalactie (ATCC BAA-611), or Bacillus cereus (ATCC 14579).

4. The Corynebacterium sp. strain according to claim 3, wherein the gene of Streptococcus mutans, the gene of Streptococcus agalactie, and the gene of Bacillus cereus have the nucleotide sequences of SEQ ID NOS. 11, 12, and 13, respectively.

5. The Corynebacterium sp. strain according to claim 2, wherein the endogenous glyceraldehyde-3-phosphate dehydrogenase-encoding gene has the nucleotide sequence of SEQ ID NO. 14.

6. The Corynebacterium sp. strain according to claim 1, wherein the Corynebacterium sp. strain is Corynebacterium glutamicum CA01-0565 (Accession No. KCCM 11013P), Corynebacterium glutamicum CA01-0566 (Accession No. KCCM 11014P), or Corynebacterium glutamicum CA01-0567 (Accession No. KCCM 11015P).

7. The Corynebacterium sp. strain according to claim 1, wherein the improved productivity of L-lysine is obtained from the reducing power increased by activation of the NADP-dependent glyceraldehyde-3-phosphate dehydrogenase.

8. A method for producing L-lysine, comprising the steps of culturing the Corynebacterium sp. Strain; and recovering lysine from the cultured cells or the culture broth,
wherein the Corynebacterium sp. strain has an activity of NADP-dependent glyceraldehyde-3-phosphate dehydrogenase and an improved productivity of L-lysine.

9. A method for producing L-lysine according to claim 8, wherein endogenous glyceraldehyde-3-phosphate dehydrogenase-encoding gene (gapA) of the Corynebacterium sp. strain is inactivated, and the exogenous NADP-dependent glyceraldehyde-3-phosphate dehydrogenase-encoding gene (gapN) is introduced to the Corynebacterium sp. strain.

10. A method for producing L-lysine according to claim 9, wherein the exogenous NADP-dependent glyceraldehyde-3-phosphate dehydrogenase-encoding gene is originated from Streptococcus mutans (ATCC 25175), Streptococcus agalactie (ATCC BAA-611), or Bacillus cereus (ATCC 14579).

11. A method for producing L-lysine according to claim 10, wherein the gene of Streptococcus mutans, the gene of Streptococcus agalactie, and the gene of Bacillus cereus have the nucleotide sequences of SEQ ID NOS. 11, 12, and 13, respectively.

12. A method for producing L-lysine according to claim 9, wherein the endogenous glyceraldehyde-3-phosphate dehydrogenase-encoding gene has the nucleotide sequence of SEQ ID NO. 14.

13. A method for producing L-lysine according to claim 8, wherein the Corynebacterium sp. strain is Corynebacterium glutamicum CA01-0565 (Accession No. KCCM 11013P), Corynebacterium glutamicum CA01-0566 (Accession No. KCCM 11014P), or Corynebacterium glutamicum CA01-0567 (Accession No. KCCM 11015P).

14. A method for producing L-lysine according to claim 8, wherein the improved productivity of L-lysine is obtained from the reducing power increased by activation of the NADP-dependent glyceraldehyde-3-phosphate dehydrogenase.
